(19) United States
(12) Patent Application Publication
(10) Pub. No.: US 2006/0211077 A1
(43) Pub. Date: Sep. 21, 2006

(54) Methods and compositions to diagnose disease using concatenated oligopeptide standard

(76) Inventors: Kenneth J. Abel, San Diego, CA (US); Andreas Braun, San Diego, CA (US); Johanna L. Whitacre, San Diego, CA (US); Kai Tang, San Diego, CA (US)

Correspondence Address: DAVID B. WALLER & ASSOCIATES, 5677 OBERLIN DRIVE, SUITE 214, SAN DIEGO, CA, 92121, US

(21) Appl. No.: 11/082,348

(22) Filed: Mar. 17, 2005

(51) Int. Cl. C12Q 1/37; G01N 33/00

(52) U.S. Cl. 435/23; 436/86

(57) ABSTRACT

The present invention is a method to quantitate proteins associated with a disease or disease state comprising the steps of isolating at least two target proteins from a test sample, combining the target proteins with a known concentration of a concatenated oligopeptide standard and a proteinase to form peptide fragments and standard peptide fragments. The concatenated oligopeptide standard comprising at least one peptide having an amino acid sequence substantially similar to a unique amino acid sequence of a peptide fragment of the two target proteins. The peptides for each of the target proteins are joined by proteinase cleavage sites to form the concatenated oligopeptide. Quantitation of the target proteins is determined by comparison of the peptide fragments to the standard peptide fragments using mass spectroscopy.

### TECHNICAL FIELD

The present invention relates generally to the measurement of protein concentrations in a sample. Specifically, the invention relates to the use of mass spectrometry to quantitatively measure the concentration of proteins in a synthetic or biological sample. More specifically, the invention relates to the use of matrix-assisted laser desorption/ionization time of flight mass spectrometry (MALDI-TOF-MS) to measure the relative and quantitative amounts of closely related protein isoforms in a biological sample.

### BACKGROUND OF THE INVENTION

The presence of disease or the magnitude of a disease state may be determined by the quantity or relative ratio of a protein specific to the disease or disease state present in a biological sample. Mass spectrometry (MS), because of its extreme selectivity and sensitivity, has become a powerful tool for the quantification of a broad range of biocatalysts including pharmaceuticals, metabolites, proteins and peptides. By exploiting the intrinsic properties of mass and charge, compounds can be resolved and confidently identified. However the signal generated by the compound will vary between runs due to differences in sample introduction, ionization process, ion acceleration, ion separation, and ion detection.

Identifying the presence of a specific protein is generally a simple process, however, quantitation of that same protein in a biological sample is often laborious and many methods that require the generation of standard protein concentration curves provide additional steps or procedures that can introduce handling or technician error. Correspondingly if there is more than one protein of interest in a sample, the errors that could result from the generation of multiple protein standard concentration curves increases substantially.

In one example, a disease known as thallassemia results from the persistent expression of γ-globin or HbF. In adults hemoglobin, or Hb, exists mainly as two α-chains (141 amino acids, 15,126 Da) and two β-chains (146 amino acids, 15,868 Da). The thalassemia syndromes, α and β thalassemias, arise from abnormal Hb function resulting from quantitative reduction in steady state levels, or total absence of α- or β-chain synthesis, respectively. While genetic analysis is underway to characterize polymorphisms and mutations that affect globin gene expression, quantification of the expressed Hb chains at the protein level would provide direct diagnosis and monitoring of the diseases.

In newborn samples, the Hb β-chain predominant in adults is not yet fully expressed. Substantial levels of fetal hemoglobin, or HbF, consisting of two α-chains and two γ-chains (Gγ and Aγ, 146 amino acids each), are still expressed at birth. The Gγ (15,995 Da) and Aγ (16,009 Da) chains differ by the presence of either glycine or alanine at amino acid 136, and are products of adjacent and highly similar genes. Around the time of birth a developmentally regulated switch is underway in which fetal γ-chains are being replaced by the adult form β chains. Coinciding with this the first year is a relative shift in the Gγ/Aγ ratio in the residual γ-chains that continue to be expressed. Whereas about 75% of the γ-chains in fetal HbF have glycine at position 136 (Gγ), the relative proportion of Gγ in the small amount of HbF in adult red cells is about 40%.

In many cases severity of adult β-thalassemia is correlated with expression levels of remaining γ-chains. Elevated levels of adult γ-globin (persistent HbF) may compensate for the reduced β-globin function, with the effect of ameliorating disease symptoms. There may exist several genetic components influencing γ-gene expression and therefore disease severity. For example, a common sequence alteration in the Gγ gene promoter, detected by the restriction endonuclease XmnI, has been implicated in regulating gene expression. The ability to rapidly characterize γ-chain expression at the protein level is important for full characterization of thalassemia disease status, and may be a useful prognostic tool in newborn screening.

Direct analysis of diluted human whole blood by protein MassARRAY technology has been successful in identifying α- and β-globin chains in normal Hb, and in thalassemia disease samples in adults. However, only the ratio of α- and β-globin chains can be obtained. Absolute quantitation is not possible in the absence of an internal standard of known quantity. HbF γ-chains are also readily detectable in newborn samples, however the relatively small mass difference (14 Da) between Gγ and Aγ makes resolution, and especially accurate quantitation of the isoforms difficult.

Consequently, there is a need in the art for a diagnostic test that utilizes an internal standard of known concentration to accurately quantitate at least two proteins of similar mass simultaneously in a sample.

### SUMMARY OF THE INVENTION

In accordance with the present invention, a method is provided to quantitate proteins associated with a disease or disease state comprising the steps of; isolating at least two target proteins from a test sample, combining said at least two target proteins with a known concentration of a concatenated oligopeptide standard and a proteinase forming peptide fragments and standard peptide fragments, said concatenated oligopeptide standard comprising at least one peptide having an amino acid sequence substantially similar to a unique amino acid sequence of a peptide fragment of said at least two target proteins, said at least one peptide for each of said at least two target proteins joined by proteinase cleavage sites to form said concatenated oligopeptide, and quantitating said peptide fragments by comparison to said standard peptide fragments using mass spectroscopy.

In another aspect of the invention a method is provided to determine the efficiency of proteinase cleavage of proteins comprising the steps of; combining a test sample containing target proteins with a known concentration of a concatenated oligopeptide standard and a proteinase forming peptide fragments and standard peptide fragments, said concatenated oligopeptide standard comprising at least one peptide having an amino acid sequence substantially similar to a unique amino acid sequence of a peptide fragment of said target proteins, said at least one peptide for each of said target proteins joined by proteinase cleavage sites to form said concatenated oligopeptide, and determining the efficiency of said proteinase cleavage using mass spectroscopy by comparison of the amount of said standard peptide fragments have been cleaved by said proteinase to the amount of said standard peptide fragments that have not been cleaved by said proteinase.

In yet another aspect of the invention a method is provided to determine proteinase activity comprising the steps of; combining a test sample containing target proteins with a known concentration of a concatenated oligopeptide standard and a proteinase for a set period of time forming peptide fragments and standard peptide fragments, said concatenated oligopeptide standard comprising at least one peptide having an amino acid sequence substantially similar to a unique amino acid sequence of a peptide fragment of said target proteins, said at least one peptide for each of said target proteins joined by proteinase cleavage sites to form said concatenated oligopeptide, and determining the activity of said proteinase said standard peptide fragments using mass spectroscopy by comparison of the amount of said standard peptide fragments that have been cleaved by said proteinase to the amount of said standard peptide fragments that have not been cleaved by said proteinase during said set period of time.

In one embodiment of these aspects the concatenated oligopeptide standard is comprised of at least one peptide fragment having an amino acid sequence substantially similar to a unique amino acid sequence of a peptide fragment from each of the at least two target proteins. The peptides are joined together by at least one proteinase cleavage site and may be joined in any order. These peptides may have the same or different amino acid sequences. The peptide fragments may have a length not less than 9 amino acids and not more than 50 amino acids. In addition, the concatenated oligopeptide may further comprise at least one isotope label such as for example ¹⁵N, ¹³C or ¹⁸O.

In the application of these methods to the quantitation of hemoglobin protein isoforms associated with thalassemias one preferred concatenated oligopeptide standard sequence is MFVSFPTTKVHVDPENFRMVTVVASALSSRTYFPHFDGSHGSAQVK (SEQ ID NO. 1), or this sequence wherein valine at position 3 is replaced with leucine, or wherein valine at position 10 is replaced with leucine, or wherein valine at position 22 is replaced with glycine or alanine or wherein glycine at position 38 is replaced with leucine. Another preferred concatenated oligopeptide standard sequence is VHVDPENFRMVTVVASALSSRTYFPHFDGSHGSAQVK (SEQ ID NO. 2) or this sequence wherein valine at position 1 is replaced with leucine or wherein valine at position 13 is replaced with glycine or alanine or wherein glycine at position 29 is replaced with leucine. Another preferred concatenated oligopeptide standard sequence is MVTVVASALSSRTYFPHFDGSHGSAQVK (SEQ ID NO. 3) or this sequence wherein valine at position 4 is replaced with glycine or alanine or wherein glycine at position 20 is replaced with leucine. Another preferred concatenated oligopeptide standard sequence is MFVSFPTTKVHVDPENFRMVTVVASALSSR (SEQ ID NO. 4) or this sequence wherein valine at position 3 is replaced with leucine, or wherein valine at position 10 is replaced with glycine or alanine, or wherein glycine at position 22 is replaced with leucine. Another preferred concatenated oligopeptide standard sequence is MFVSFPTTKVHVDPENFR (SEQ ID NO. 5) or this sequence wherein valine at position 3 is replaced with leucine or wherein valine at position 10 is replaced with glycine or alanine.

In another embodiment the peptide fragments that may be joined to prepare a concatenated peptide standard for use in the methods of the present invention preferably include the following sequences MVTVVASALSSR (SEQ ID NO. 6), VHVDPENFR (SEQ ID NO. 7), TYFPHFDLSHGSAQVK (SEQ ID NO. 8) and MFVSFPTTK (SEQ ID NO 9).

In yet another embodiment the unique peptide sequences of the target proteins from which the peptide sequences of the concatenated oligopeptide are modeled include MVTGVASALSSR (SEQ ID NO. 10), MVTAVASALSSR (SEQ ID NO. 11), LHVDPENFR (SEQ ID NO. 12), TYFPHFDGSHGSAQVK (SEQ ID NO. 13) and MFLSFPTTK (SEQ ID NO. 14).

In another embodiment the proteinase cleavage sites that may be used to join the peptides that form the concatenated oligopeptide standard may be for example Lyc-C, trypsin, staphylococcus aureas V8 or Asp-N.

In yet another embodiment the at least two target proteins may be for example antibodies, hemoglobin proteins, glycoprotein or enzymes.

In still another embodiment the mass spectrometry used in conjunction with the concatenated oligopeptide standard to quantitate the peptide fragments and target proteins is MALDI TOF mass spectrometry.

### BRIEF DESCRIPTION OF THE DRAWINGS

FIG. 1: is a chromatograph of a an equal concentration of concatenated oligopeptide standard and hHb digested overnight with 0.4 ug trypsin (1:25 enzyme:protein).

FIG. 2: is a chromatograph of a an equal concentration of concatenated oligopeptide standard and hHb digested overnight with 1.0 ug trypsin (1:25 enzyme:protein).

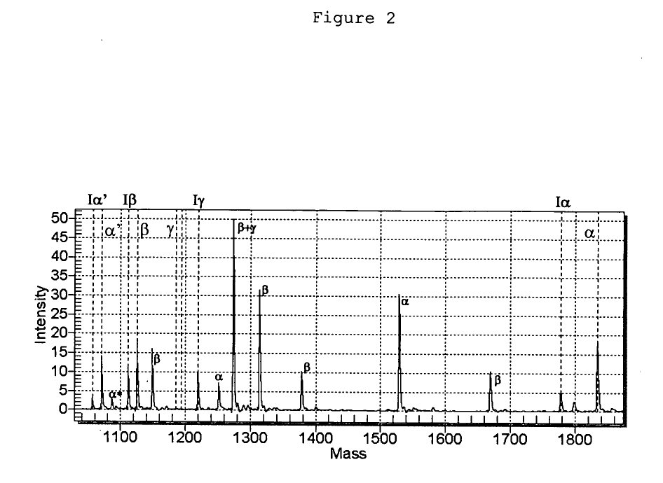

FIG. 3: is a chromatogram of a digested concatenated oligopeptide standard of the present invention.

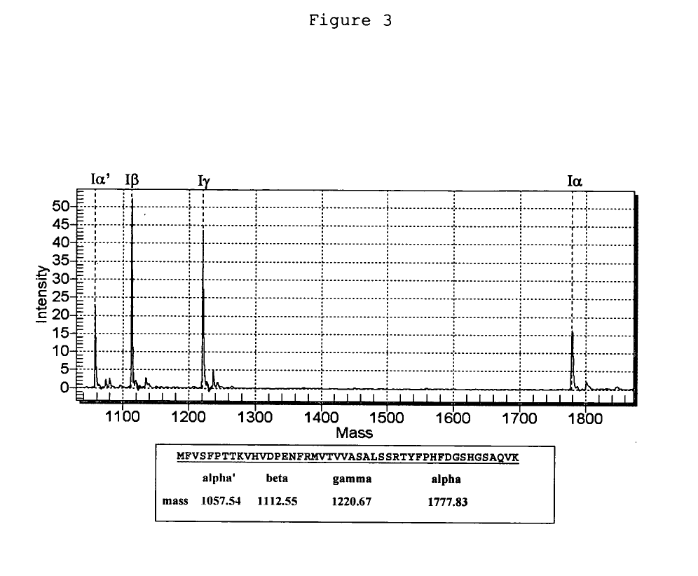

FIG. 4: A and B are MALDI-TOF mass spectra of different spottings of the same sample on a chip demonstrating the lack of reliable quantification of signature target peptides due to the varying relative peak intensities between spots within a chip and between sample preparations.

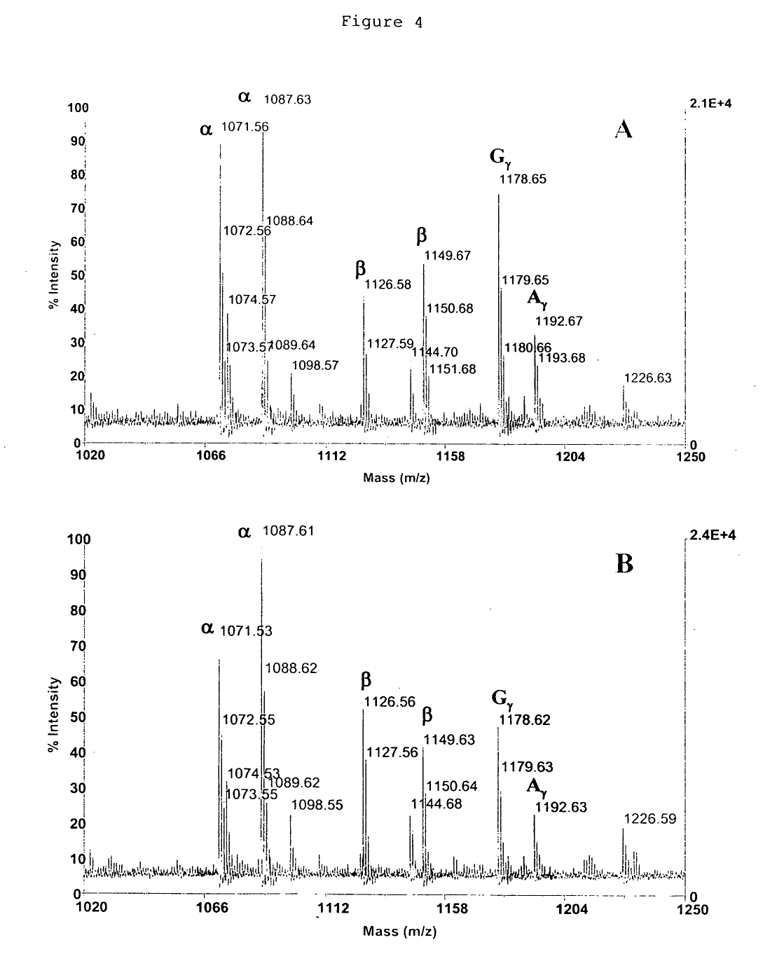

### DETAILED DESCRIPTION

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information is known and can be readily accessed, such as by searching the Internet and/or appropriate databases. Reference thereto evidences the availability and public dissemination of such information.

As used herein, the term “substantially similar” refers to an amino acid sequence of the standard peptides that are joined by proteinase cleavage sites to form the concatenated oligopeptide standard in reference to the specific unique peptide fragment sequence of the target protein. More specifically, substantially similar means that the amino acid sequence of each standard peptide is independently 100% identical to a unique peptide fragment of a target protein, or each standard peptide amino acid sequence is about 95% identical to a unique peptide fragment of a target protein, or each standard peptide amino acid sequence is about 90% identical to a unique peptide fragment of a target protein, or each standard peptide amino acid sequence is about 85% identical to a unique peptide fragment of a target protein, or each standard peptide amino acid sequence is about 80% identical to a unique peptide fragment of a target protein, or each standard peptide amino acid sequence is about 75% identical to a unique peptide fragment of a target protein, or each standard peptide amino acid sequence is about 70% identical to a unique peptide fragment of a target protein, or each standard peptide amino acid sequence is about 65% identical to a unique peptide fragment of a target protein, or each standard peptide amino acid sequence is about 60% identical to a unique peptide fragment of a target protein.

As used herein a “unique peptide fragment(s)” or “signature target peptide(s)” or “target peptide” of a target protein refers to an amino acid sequence of a peptide fragment generated from proteinase digestion of a target protein in a test sample that cannot be associated with any other protein present in that test sample.

As used herein the term “standard peptide” or “peptide standard” refers to an amino acid sequence that is substantially similar to a unique peptide fragment of a target protein generated from proteinase digestion of the target protein.

As used herein, a “target protein(s)” is a protein that is examined using the methods disclosed herein. Examples of target proteins include enzymes, transport proteins such as hemoglobin, structural proteins such as myosin, immunological proteins such as antibodies, neurological proteins such as rhodopsin and proteins involved in cell growth and differentiation such as repressor proteins. The intended target will be clear from the context of the invention.

As used herein, “treat” or “treating” refers to the process of exposing the protein or concatenated oligopeptide standard to conditions under which modification occurs. For example, exposure to proteinase that cleaves the protein and concatenated oligopeptide at specific sites within the molecules forming protein fragments and standard peptide fragments.

As used herein the phrase “mass spectrometric analysis” refers to the determination of the charge to mass ratio of atoms, molecules or molecule fragments.

As used herein, signal, peak, or measurement in the context of a mass spectrum and analysis thereof refers to the output data, which can reflect the charge to mass ratio of an atom, molecule or fragment of a molecule, and also can reflect the amount of the atom, molecule, or fragment thereof, present. The charge to mass ratio can be used to determine the mass of the atom, molecule or fragment of a molecule, and the amount can be used in quantitative or semi-quantitative methods. For example, in some embodiments, a signal peak or measurement can reflect the number or relative number of molecules having a particular charge to mass ratio. Signals or peaks include visual, graphic and digital representations of output data.

As used herein, comparing measured masses or mass peaks refer to analyzing one or more measured sample mass peaks to one or more sample or reference mass peaks.

As used herein, a reference mass generated from a standard peptide is a mass with which a measured sample mass generated from a unique amino acid sequence peptide fragment of a target protein can be compared. The amino acid sequence of the standard peptide is substantially similar to the amino acid sequence of the target peptide consequently the their observed masses are similar in magnitude but not identical. Because the concatenated oligopeptide is assembled from peptide of known amino acid sequence and joined by proteinase cleavage sites the reference masses obtained from digestion of the oligopeptide are known. However, a reference mass for a given peptide can be calculated, can be present in a database or can be experimentally determined.

As used herein, “fragmentation” or “cleavage” refers to a procedure or conditions in which a target protein or concatenated oligopeptide standard molecule is severed into two or more smaller peptide fragments. Such fragmentation or cleavage is preferably sequence specific and can be accomplished by any of a variety of methods, reagents or conditions, including, for example, chemical, enzymatic, physical fragmentation.

As used herein, fragmentation conditions refer to chemical, enzymatic or physical conditions under which fragmentation or cleavage of a target protein and/or concatenated oligopeptide standard can be achieved. Cleavage conditions or cleavage reaction conditions can include one or more cleavage reagents that are used to perform the cleavage reactions, and other parameters of the reactions including, but not limited to, time, temperature, pH, or choice of buffer or ions. For example, fragmentation conditions can refer to buffer containing sequence-specific proteinase, where specific amino acid sequences of the target protein and/or concatenated oligopeptide standard are recognized by the proteinase and cleaved, and other areas in these molecules that do not contain these recognized amino acid sequences are not cleaved.

As used herein, “peptide fragment(s)”, “target peptide fragment(s)” and “standard peptide fragment(s)” refer to peptides resultant from a fragmentation or cleavage of a target protein or concatenated oligopeptide standard.

As used herein the term “complete cleavage” or “total cleavage” refers to a cleavage reaction in which all the cleavage sites recognized by a particular cleavage reagent are cut to completion.

As used herein, matrix or support particles refers to support materials that are in the form of discrete particles. The particles have any shape and dimensions, but typically have at least one dimension that is 100 mm or less, 50 mm or less, 10 mm or less, 1 mm or less, 100 μm or less, 50 μm or less, 10 μm or less, 5 μm or less, 1 μm or less, 0.5 μm or less, and typically have a size that is 100 mm³ or less, 50 mm³ or less, 10 mm3 or less, and 1 mm³ or less, 100 μm³ or less and can be on the order of cubic microns; typically the particles have a diameter of more than about 1.5 microns and less than about 15 microns, such as about 4-6 microns.

As used herein, “substrate” refers to an insoluble support that can provide a surface on which or over which a reaction can e conducted and/or a reaction product can be retained at identifiable loci. Support can be fabricated from virtually any insoluble or solid material. For example, silica gel, glass (e.g., controlled-pore glass (CPG)), nylon, Wang resin, Merrifield resin, Sephadex, Sepharose, cellulose, a metal surface (e.g., polyethylene, polypropylene, polyamide, polyester, polyvinylidenedi-fluoride (PVDFi). Exemplary substrates include, but are not limited to flat supports such as glass fiber filters, glass surfaces, metal surfaces (steel, gold, silver, aluminum, copper and silicon), and plastic materials. The solid support is in any form suitable for mounting ion the cartridge base, including, but not limited to: a plate, membrane, wafer, a wafer with pits, a porous three-dimensional substrate, and other geometries and forms known to those of skill in the art. Exemplary supports are flat surfaces designed to receive or link samples at discrete loci, such as flat surfaces with hydrophobic regions surrounding hydrophilic loci for receiving, containing or binding a sample.

As used herein, “a” refers to one or more.

As used herein, “test sample” refers to a composition containing a target protein to be detected. The test sample may be a synthetic sample or a biological sample and may contain a concatenated oligopeptide standard. A test sample may be obtained from a living source, for example, an animal, preferably a mammal and most preferably a human. A biological sample can be in any form, including a solid material such as a tissue, cells, a cell pellet, a cell extract, or a biopsy or a biological fluid such as urine, blood, interstitial fluid, peritoneal fluid, plasma, lymph, ascites, sweat, saliva, follicular fluid, breast milk, non-milk breast secretions, serum, cerebral spinal fluid, feces,-seminal fluid, lung sputum, amniotic fluid, exudates from a region of infection or inflammation, a mouth wash containing buccal cells, synovial fluid, or any other fluid sample produced by the subject. In addition, the sample can be solid samples of tissues or organs, such as collected tissues, including bone marrow, epithelium, stomach, prostate, kidney, bladder, breast, colon, lung, pancreas, endometrium, neuron, muscle, and other tissues. If desired, solid materials can be mixed with a fluid or purified or otherwise treated. Samples examined using the methods described herein can be treated in one or more purification steps in order to increase the purity of the desired proteins in the sample. In particular, herein, the samples include a mixture of matrix used for mass spectrometric analyses and a biopolymer, such as a target protein and/or a concatenated oligopeptide standard.

As used herein, a cellular extract refers to a preparation or fraction that is made from a lysed or disrupted call.

The novel methods and compositions of the present invention may be used to assist in the diagnosis and treatment of medical conditions such as heart failure. In diagnosing this condition it is important to measure, or quantitate, the amounts of specific key proteins in samples where multiple distinct proteins are present. MALDI-TOF. MS methods have been used to accurately measure the amounts of proteins in these types of samples. As an example, α-MyHC mRNA expression is down regulated in heart failure and β-MyHC mRNA expression is up regulated. These changes are reversed in-patients successfully treated with adrenergic receptor blockers. This suggests that changes in MyHC protein expression are important for cardiac function, and provide a useful diagnostic and prognostic indicator. The isoforms are highly homologous and very difficult to distinguish by conventional means, yet are quite amenable to evaluation by the present invention. The amounts of α- and β-MyHC protein have been determined both relative to each other and with regard to absolute amounts of these related species (see patent application serial no.: PCT/US2003/034386 (WO 2004/042072) incorporated herein in its entirety).

In addition the novel methods and compositions of the present invention may be used to diagnose or monitor diseases such as thalassemia. This disease results from the persistent expression of γ-globin or HbF. In adults hemoglobin, or Hb, exists mainly as two α-chains (141 amino acids, 15,126 Da) and two β-chains (146 amino acids, 15,868 Da). The thalassemia syndromes, α and β thalassemias, arise from abnormal Hb function resulting from quantitative reduction in steady state levels, or total absence of α- or β-chain synthesis, respectively.

In newborn samples, the Hb β-chain predominant in adults is not yet fully expressed. Substantial levels of fetal hemoglobin, or HbF, consisting of two α-chains and two γ-chains (Gγ and Aγ, 146 amino acids each), are still expressed at birth. The Gγ (15,995 Da) and Aγ (16,009 Da) chains differ by the presence of either glycine or alanine at amino acid 136, and are products of adjacent and highly similar genes. Around the time of birth a developmentally regulated switch is underway in which fetal γ-chains are being replaced by the adult form β chains. Coinciding with this the first year is a relative shift in the Gγ/Aγ ratio in the residual γ-chains that continue to be expressed. Whereas about 75% of the γ-chains in fetal HbF have glycine at position 136 (Gγ), the relative proportion of Gγ in the small amount of HbF in adult red cells is about 40%.

In many cases severity of adult β-thalassemia is correlated with expression levels of remaining γ-chains. Elevated levels of adult γ-globin (persistent HbF) may compensate for the reduced β-globin function, with the effect of ameliorating disease symptoms.

The present invention utilizes a concatenated oligopeptide standard comprising peptides joined by proteinase cleavage sites that have amino acid sequences substantially similar to unique proteinase cleavage fragments of the target proteins in a test sample. When cleaved these standard peptide fragments will produce MALDI-TOF MS signals that are proportional to the relative concentrations of those peptides, and thus can be used as accurate and sensitive internal standards for quantitation of the target proteins. This relationship holds for both linear and reflector modes of MALDI-TOF MS, as well as when signals are measured by peak intensity or peak area.

A. Design of Concatenated Oligopeptide Standard

A wide variety of proteins may be selected and quantitated using the methods and compositions of the present invention. Preferably the target protein is present in a sufficient quantity in a biological sample so that it may be quantitated by mass spectral analysis without further manipulation. However, if required the protein may be concentrated using standard methods known in the art prior to preparation for mass spectral analysis. Proteins that may be detected include but are not limited to endogenous proteins such as hemoglobin and myosin, exogenous proteins such as those present in a test sample due to infection such as viral or bacterial proteins or proteins produced in response to disease such as antibodies.

When using the methods and compositions for detecting the presence or absence of a disease or disease state the target proteins selected are those associated with the disease of disease state. Alternatively, when using the methods and compositions of the present invention for determining the cause, or extent, of an infection the target proteins selected are those produced by the agent causing the infection such as a viral coat protein or a protein produced in response to the infection such as an antibody.

Reference peptides for a protein to be detected are selected based on one or more unique “signature” amino acid sequence(s) specific to that protein in a sample. A highly conserved protein such as human cardiac α-myosin heavy chain (α-MyHC) would have diagnostic peptides shared with other species, but if only human samples were to be analyzed, then the diagnostic peptide would only have to discriminate human cardiac α-MyHC from other human cardiac myosin isoforms. The selection of the diagnostic peptide thus sets the parameters for the design of the standard peptide.

The standard peptide is highly homologous to the diagnostic peptide thus, the sequence of the diagnostic peptide is the starting point for the design of the standard peptide. The sequence is preferably altered to change the mass of the standard peptide so it can be discriminated from the reference peptide by MALDI-TOF MS while maintaining the chemistry of the original reference peptide. This maybe achieved by a single conservative amino acid substitution (such as the substitution of valine for isoleucine) allowing for the standard peptide to be easily prepared with standard solid phase peptide synthesizers. A variety of amino acids may be utilized with the present invention including unusual amino acids, non-natural enatiomers of commonly occurring amino acids or stable isotope amino acids. The substitution preferably does not change the charge or hydrophobicity of the peptide because this might alter the recovery of the peptide, the ability of the peptide to co-crystallize with matrix or the ability to ionize, which could potentially change the production of its MALDI-TOF signal. Preferably the standard peptide has a MALDI-TOF MS mass signal that does not overlap with any other peptide present in the sample. This may become more difficult as the complexity of the sample increases.

In myocardial infarct there is a change in the α- and β-MyHc protein concentrations in the body. The magnitude of this change over time is important for effective treatment following the attack. The sequences of α- and β-MyHC were examined following digestion to find a pair of tryptic peptides, one from each isoform that would be suitable for MALDI-TOF MS quantification. Suitable peptides are preferably similar in sequence, discriminated by mass, and generate a strong MALDI-TOF ion current. Ideally the peptides result from the cleavage of trypsin sites that are homologous between the isoforms so that one isoform is not cleaved in preference to the other isoform. It is preferable that the peptides be similar in sequence so that their recovery, crystallization with matrix, and ionization by MALDI be equivalent. This may be achieved by a single conservative amino acid substitution. A search of the sequences revealed ˜10 pairs of tryptic peptides fitting these criteria.

A one dimensional gel electrophoresis produced a cardiac myosin heavy chain sample with a MALDI-TOF spectra that had an open region in which the standard peptide signal could appear without interference from other peptides. Other methods that maybe used to produce a sample with a MALDI-TOF spectra that has an open region in which the standard peptide signal can appear without interference from other peptides include two dimensional electrophoresis and immuno-precipitation. This open region is preferably in close proximity to the reference peptide since the standard peptide will have a mass close to that of the reference peptide. If there are several potential reference peptides for a given protein, then the sample spectra can be inspected to find the reference peptides that have the highest signal and that have open regions for the standard peptide signal nearby. For any given protein and/or sample, the MALDI-TOF spectra may be analyzed to select the optimal reference peptides, that may be used to design optimal standard peptides by the procedures described above. In one preferred method peptides peptide fragments showing high intensity on MALDI-TOF spectra having the least amount of variability relative to other peaks from experiment to experiment or from one spotting to the next were selected. Using this technique a unique peptide amino acid sequence was selected for both α- and β-MyHC. Their sequences and monoisotopic mass is provided below.

| | | |
|---|---|---|
| Name | Sequence | M + H (monoisotope) |

| | | |
|---|---|---|
| α-MyHC | ILNPVAIPEGQFIDSR | 1768.96 |
| | (SEQ ID NO. 15) | |
| β-MyHC | ILNPAAIPEGQFIDSR | 1740.93 |
| | (SEQ ID NO. 16) | |

Each of these unique target peptide amino acid sequences were modified by replacing the isoleucine at position 7 of with valine (shown in bold type). These peptides are then joined by a proteinase cleavable site, in this case a trypsin cleavage site to form a concatenated oligopeptide. The oligopeptide standard that may be used for the quantitation α- and β-myosin sequence is:

ILNPVAVPEGQFIDSR|ILNPAAVPEGQFIDSR (SEQ ID NO. 17)

The bar between the arginine, residue 16, and the isoleucine, residue 17 indicates the trypsin cleavage site.

Another highly conserved protein is hemoglobin. Diseases associated with expression levels of specific types of hemoglobin such as thalassemia may be quantitated with the methods and compositions of the present invention.

With the exception of the one amino acid difference between Gγ and Aγ, it is difficult to identify homologous tryptic peptide fragments resulting from digestion of the human globin chains (α, β, Gγ and Aγ) that differ by a single amino acid. The unique peptide amino acid sequences selected for α, β, Gγ and Aγ globins and their monoisotopic masses are provided below.

| | | M + H |
|---|---|---|
| Name | Sequence | (monoisotope) |
| Gγ | MVTGVASALSSR | 1178.61 |
| | (SEQ ID NO. 10) | |
| Aγ | MVTAVASALSSR | 1192.64 |
| | (SEQ ID NO. 11) | |
| β | LHVDPENFR | 1126.56 |
| | (SEQ ID NO. 12) | |
| α | TYFPHFDLSHGSAQVK | 1833.89 |
| | (SEQ ID NO. 13) | |
| α′ | MFLSFPTTK | 1071.55 |
| | (SEQ ID NO. 14) | |

In order to quantify the four different chains, three standard peptides were developed one for each protein that is highly similar to peptides in α- and β-chains, and a third for the peptide pair distinguishing Gγ and Aγ. In this example of the present invention two alpha peptide fragments were selected. The alpha peptide having its mass peak at 1834 Da showed the least variability and was the most stable alpha peak but had a mass peak substantially separated from the other selected peaks on the MALDI-TOF spectra. Consequently a second peak at 1071 Da, closer to the other observed peaks was selected. In addition, having two alpha peaks allowed comparison measurements to validate and insure accuracy of the signals.

Each unique target peptide amino acid sequence was modified by replacing a single amino acid that does not change the charge or hydrophobicity of the original peptide. The standard peptide developed for use in the quantitation of Gγ and Aγ replaces the glycine and alanine (residue 4) respectively with valine. Similarly leucine (residue 1) was replaced with valine to create the peptide standard for β-globin, glycine replaced leucine (residue 8) to create the standard peptide for α-globin and a second peptide standard for α-globin replaced leucine (residue 3) with valine. These standard peptide sequences, their monoisotopic mass and neighboring peaks are shown below.

| | | | |
|---|---|---|---|
| Name | Sequence | M + H (monoisotope) | Neighboring Peaks |

| | | | |
|---|---|---|---|
| Iγ | MVTVVASALSSR | 1220.67 | 1149β, 1171α*, 1252α |
| | (SEQ ID NO. 6) | | |
| Iβ | VHVDPENFR | 1112.54 | 1098γ, 1144γ*, 1149B + γ |
| | (SEQ ID NO. 7) | | |
| Iα | TYFPHFDGSHGSAQVK | 1777.83 | 1738γ*, 1798β*, 1866β |
| | (SEQ ID NO. 13) | | |
| Iα′ | MFVSFPTTK | 1057.53 | 1017γ, 1088α |
| | (SEQ ID NO. 14) | | |

The peptides are joined by proteinase cleavage sites to avoid the cumbersome and error prone steps of quantifying and normalizing each internal standard separately for absolute quantification of the target protein(s). These sites allow the oligopeptide standard to be digested into its corresponding peptide fragments that are substantially similar in their amino acid sequence to a unique target protein peptide sequence. Below is one example of a synthetic concatenated oligopeptide standard for detection of thalassemia:

(SEQ ID NO. 1)

MFVSFPTTK|VHVDPENFR|MVTVVASALSSR|TYFPHFDGSHGSAQVK

The bars indicate the trypsin cleavage sites.

The ordering of the peptide fragments in a concatenated oligopeptide may be determined by a variety of elements. In this example the arrangement of the fragments in the oligopeptide was determine based on the ease of synthesis. More particularly, minimization of hydrophobic residues near the C-terminus simplified synthesis and purification.

Quantitation of a target protein is performed by mixing the concatenated oligopeptide standard with a biological sample (e.g. dilute whole blood). The mixture is digested using a proteinase (e.g. endoproteinase trypsin) that is specific for the cleavage sites incorporated between the peptides in the oligopeptide standard. Concurrent with digestion of the hemoglobin chains, complete digestion releases the individual peptide standards in equimolar amounts. Therefore quantification is performed once for the concatenated oligopeptide standard, and normalization of the released internal peptide standards is achieved automatically. The addition of the concatenated oligopeptide standard to the reaction mixture before proteinase digestion also provides an indicator of digest efficiency as peaks corresponding to partial digest products can be detected.

For quantitation of proteins associated with thalassemia using the concatenated oligopeptide above the anticipated partial digested fragments and their monoisotopic mass are provided below.

| | Fragment | |
|---|---|---|
| Position | [M + H]⁺ | Sequence |
| 1-9 | 1057.54 | MFVSFPTTK |
| | | (SEQ ID NO. 9) |
| 10-18 | 1112.55 | VHVDPENFR |
| | | (SEQ ID NO. 7) |
| 19-30 | 1220.67 | MVTVVASALSSR |
| | | (SEQ ID NO. 6) |
| 31-46 | 1777.83 | TYFPHFDGSHGSAQVK |
| | | (SEQ ID NO. 8) |
| 1-18 | 2151.07 | MFVSFPTTKVHVDPENFR |
| | | (SEQ ID NO. 5) |
| 10-30 | 2314.20 | VHVDPENFRMVTVVASALSSR |
| | | (SEQ ID NO. 18) |
| 19-46 | 2979.48 | MVTVVASALSSRTYFPHFDGSHGSAQVK |
| | | (SEQ ID NO. 3) |
| 1-30 | 3352.72 | MFVSFPTTKVHVDPENFRMVTVVASALSSR |
| | | (SEQ ID NO. 4) |
| 10-46 | 4073.01 | VHVDPENFRMVTVVASALSSRTYFPHFDGSHGSAQVK |
| | | (SEQ ID NO. 2) |
| 1-46 | 5111.53 | MFVSFPTTKVHVDPENFRMVTVVASALSSRTYFPHFDGSHGSAQVK |
| | | (SEQ ID NO. 1) |

Specific and unique amino acid sequences of particular target proteins may be identified on computerized databases known to those of ordinary skill in the art that provide complete or partial sequence of a number of known proteins. One such database is the National Center for Biotechnology Information's Genbank and GenPept databases (www.ncbi.nlm.nih.gov). Alternatively target proteins may be sequenced and these sequences scanned manually to select unique and characteristic sequences that may be synthesized for the preparation of a concatenated oligopeptide standard.

In certain embodiments the size of each peptide that is combined to prepare the concatenated oligopeptide standard may comprise, but is not limited to not less than about 9 and not more than about 50 amino acid residues. More specifically the peptide may comprise about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50 amino acid residues.

As stated above, the sequence of residues of the concatenated oligopeptide standard peptides may be interrupted by one or more non-amino molecule moieties. For example one or more peptides of the concatenated oligopeptide standard may include one or more modified or unusual amino acid residues, such as for example norvaline, norleucine, ornithine, N-methyllysine, N-methylisoleucine, hydroxyproline or hydroxylysine.

A variety of proteinase cleavage sites may be incorporated into the concatenated oligopeptide standard. Proteinases that cleave consistently and predictably at a particular site such as between two specific amino acids are preferred. In view of this preference the design of each peptide that is joined to form a concatenated oligopeptide must have terminal amino acids that when joined to another standard peptide provide a cleavage site appropriate for the proteinase selected. Preferred proteinases include for example Lyc-C, trypsin, staphylococcus aureasV8 and Asp-N.

A number of peptide sequences may be bound by proteinase cleavage sites to form the concatenated oligopeptide. The overall length of the concatenated oligopeptide may be a limiting factor when joining a number of these peptides. More specifically the longer the concatenated oligopeptide becomes the greater the opportunity for the oligopeptide to fold into structures that might interfere with the efficiency of cleavage. Preferably the oligopeptide is not less than two peptides joined by a single proteinase cleavage site to not more than ten peptides.

The length of the concatenated oligopeptide will depend on the number of peptides that are joined to form the oligopeptide and the amino acid sequences of those peptides. The peptides vary in length from not less than nine amino acids residues to not more than 50 amino acids residues. Consequently, the concatenated oligopeptide is not less than 18 amino acid residues in length and not more than 100 amino acids.

An example of a method for chemical synthesis of a concatenated oligopeptide standard is as follows. Using the solid phase peptide synthesis method of Sheppard et al. (1981) an automated peptide synthesizer (Pharmacia LKB Biotechnology Col., LKB Biotynk 4170) adds N,N′-dicyclohexylcarbodiimide to amino acids hose amine functional groups are protected by 9-fluorenylmethoxycarbonyl groups, producing anhydrides of the desired amino acid (FMOC-amino acids). An FMOC amino acid corresponding to the C-terminal amino acid of the desired peptide is affixed to Ultrosyn A resin (Pharmacia LKB Biotechnology Co.) through its carboxyl group, using dimethylaminopyridine as a catalyst. The resin is then washed with dimethylformamide containing piperidine resulting in the removal of the protective amine group of the C-terminal amino acid. An FMOC-amino acid anhydride corresponding to the next residue in the peptide sequence is then added to the substrate and allowed to couple with the unprotected amino acid affixed to the resin. The protective amine group is subsequently removed from the second amino acid and the above process is repeated with additional residues added to the peptide in a like manner until the sequence is completed. After the peptide is completed, the protective groups, other than the acetoamidomethyl group are removed and the peptide is released from the resin with a solvent consisting of, for example, 94% (by weight) trifluroacetic acid, 5% phenol, and 1% ethanol. The synthesized peptide is subsequently purified using high-performance liquid chromatography or other peptide purification techniques such as for example, thin layer chromatography, gas chromatography, paper chromatography, affinity chromatography or supercritical flow chromatography.

B. MALDI-TOF MS

While there have been reports of quantitative MALDI-TOF MS applications, there are many problems inherent to the MALDI ionization process that have restricted its widespread use (Kasmaier et al., 1998; Horak et al., 2001; Gobom et al., 2000; Wang et al, 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF MS to on-line separation techniques such as liquid chromatography. Combined, these factors are thought to compromise the accuracy, precision, and utility with which quantitative determinations can be made.

Semi-quantitative MALDI mass spectrometric results have been reported with nanoliter volumes of sample loaded onto chips. The area under the peaks in resulting mass spectra is proportional to the relative concentrations of the components of the sample. Methods for preparing and using such chips are known in the art, as exemplified in U.S. Pat. No. 6,024,925, U.S. Publication 2001 0008615 and PCT Application No. PCT/US97/20195 (WO 98/20020); methods for preparing and using such chips also are provided in co-pending U.S. application Ser. Nos. 08/786,988, 09/364,774, and 09/297,575. Chips and kits for performing these analyses are commercially available from SEQUENOM (San Diego, Calif.) under the trademark MassARRAY™.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Yang et al., 2000; Wittmann et al., 2001), whereas other studies have demonstrated the potential of MALDI-TOF-MS for the quantification of biologically relevant analytes such as neuropathies, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000).

The properties of the matrix material used in the MALDI method is important and only a select group of compounds is useful for the selective desorption of proteins and polypeptides. A review of all the matrix materials available for peptides and proteins shows that there are certain characteristics the compounds must share to be analytically useful. The materials that work well are used heavily by all MALDI practitioners and new molecules are constantly being evaluated as potential matrix candidates. With a few exceptions, most of the matrix materials used are solid organic acids. Liquid matrices have been investigated, but are not used routinely.

I. Sample Preparation

In general, all reasonable efforts should be made to reduce excessive contamination in the samples. Always use the best quality solvents, reagents and samples. HPLC-grade solvents should be the standard in MALDI experiments. Samples are preferably maintained in plastic containers.

Optimum sample handling conditions for biological preparations usually involve non-volatile salts. Consequently, desalting may be recommended to reduce excessive categorization, increase resolution or decrease signal suppression. Washing the analyte-doped matrix crystals with cold acidic water has been suggested as a very efficient way of desalting samples that have already been crystallized with the matrix. However, whenever possible, it is best to remove the salts, before the crystals are grown, using some of the techniques described later. There is a competition between protonation and categorization in MALDI when salts are present, and the choice between the two processes is still the subject of investigation.

When working with complex biological materials in MALDI it is often necessary to use detergents, otherwise the proteins, especially at less than millimeter concentrations, will be rapidly absorbed on accessible surfaces and effectively suppress protein peaks in the spectrum. The effect of detergents on MALDI spectra depends on the type of detergent and sample.

Nonionic detergents such as TritonX-100, Triton X-114, N-octylglucoside and Tween 80 generally do not interfere significantly with sample preparation. These detergents are often required for the analysis of hydrophobic proteins and can provide good internal calibration peaks in the low mass range of the mass spectrum. For example, it has even been reported that Triton X-100, in a concentration up to 1%, is compatible with MALDI and in some cases it can improve the quality of spectra. N-octylglucoside has been shown to enhance the MALDI-MS response of the larger peptides in digest mixtures.

Ionic detergents and particularly sodium dodecyl sulfate (SDS), can severely interfere with MALDI even at very low concentrations. Consequently concentrations of SDS above 0.1% must be reduced by sample purification prior to crystallization with the matrix. This is particularly important given the wide application of MALDI to the analysis of proteins separated by SDS-PAGE. Once a protein has been coated with SDS, simply removing the excess SDS from the solution will not improve sample prep for MALDI: the SDS shell formed around the protein must also be removed. Typical purification schemes involve two phase extraction such as reversed-phase chromatography or liquid-liquid extraction.

Involatile solvents such as glycerol, polyethylene glycol, β-mercaptoethanol, dimethylsulfoxide (DMSO) and dimethylformamide (DMF) are often used in protein chemistry. These solvents interfere with matrix crystallization and coat any crystals that do form with a solvent layer that is difficult to remove. If these solvents are used and the dried-droplet method does not yield good results, a different crystallization technique such as crushed-crystal method should be considered.

The use of buffers is often utilized in protein sample preparation to maintain biological activity and structural or conformational integrity. In cases where buffers are possible sources of interference, increasing the matrix:analyte ratio has shown promise. The effect of six common buffer systems, on the MALDI spectra of bovine insulin, cytochrome C and bovine albumin with DHB as a matrix has been studied (Wilkins et al., 1998). In general MALDI mass spectral analysis is tolerant of buffers.

In order to obtain samples free of contaminants that interfere with MALDI analysis, i.e. free of salts, buffers, detergents and in volatile compounds, several experimental approaches have been tested with varying results. A number of investigators have attempted to establish “MALDI from synthetic membranes” as a general purification tool in protein biochemistry. In an extensive series of experiments, analyte droplets were deposited on to polymeric membranes (porous polyethylene, polypropylene, analyte, nylon, Nafion, and others), washed in special solvents, and mixed with matrix to provide “clean” crystals this approach has proven to be useful for the direct analysis of proteins electro blotted from SDS-PAGE gels into synthetic membranes. In another approach, protein samples were desalted and freed of salts and detergents by constructing self-assembled monolayers of octadecylmercaptan (C18) on a gold coated MALDI probe surface. These surfaces were able to reversibly bind polypeptides through hydrophobic interactions allowing simultaneous concentration and desalting of the analyte.

Surface enhanced affinity capture (SEAC) was created (Hutchens et al., 1993) to facilitate the desorption of specific macromolecules affinity-captured directly from unfractionated biological fluids and extracts. This technique has also been successfully used as a means for sample purification. Direct analysis of affinity-bound analytes by MALDI-TOF is now performed routinely and it is even possible to get customized affinity-capture sample probes from commercial sources.

Purification of analyte samples by traditional methods, such as alcohol or acetone precipitation, HPLC, ultra filtration, liquid-liquid extraction, dialysis and ion exchange are always recommended; however, the effects of increased sample preparation time and sample recovery yields must be considered carefully. It is possible to purify samples prior to analysis by using small, commercially available C18 reverse-phase micro columns or centrifugal ultra filtration devices; however, such devices can still suffer from the same drawbacks as large scale separation schemes.

The degradation of signal intensity and resolution that results from excessive contamination can sometimes be eliminated by more extensive dilution of the protein in the matrix solution, a commonly used method is to prepare a 1:5 dilution series of the sample. Diluting the protein solution often improves the MALDI signal.

II. Matrix Selection

Solubility in commonly used protein solvent mixtures is one characteristic of an optimal matrix. To incorporate the protein or peptide (target and/or standard) into a growing matrix crystal requires that the protein and the matrix be simultaneously in solution. Therefore, a matrix should dissolve and grow protein-doped crystals in commonly used protein-solvent systems. More specifically, any solvent system in which the analyte of interest will co-dissolve with the matrix may be appropriate mixture to support protein doped matrix crystals. In practical terms, this means that the matrix must be sufficiently soluble to make 1-100 mM solutions in solvent systems consisting of: acidified water, water-acetonitrile mixtures, water-alcohol mixtures, 70% formic acid, etc.

The light absorption spectrum of the matrix crystals preferably overlaps the frequency of the laser pulse being used so that the laser pulse energy is deposited in the matrix. Unfortunately the absorption coefficients of solid systems are not easily measured and are usually red shifted (Stokes shift) relative to the values in solution. The extent of the shifts varies from compound to compound. The solution absorption coefficients are often used as a guide, and typical ranges for commonly used matrix materials, at the wavelengths they are applied, are e=3000'16000 (1 mol−1 cm−1). UV-MALDI, with compact and inexpensive nitrogen lasers operating at 337 nm is the most common instrumental option for the routine analysis of peptides and proteins. IR-MALDI of peptides has been demonstrated but is not used in analytical applications. For UV-MALDI, compounds such as some trans-cinnamic acid derivatives (CHCA) and 2,5-dihydroxy benzoic acid (DHB) have proven to give the best results.

At appropriate matrix to analyte mixing ratios, small to moderately sized analyte ions (1000-20000 Da) can fully suppress positively charges matrix ions in MALDI mass spectra. This is true for all matrix species, and is observed regardless of the preferred analyte ion form (protonated or cationized). Since the effect has been observed with a number of matrices including CHCA and DHB, it seems to be a general phenomenon in MALDI. Along with the fact that fragmentation is weak in MALDI, this leads to nearly ideal mass spectra with a strong peak for the analyte ions and no other signals present.

Several additives have been added to MALDI samples to enhance the quality of the mass spectra. Additives, also known as co-matrices, can increase the homogeneity of the matrix/analyte deposit; decrease/increase the amount of fragmentation; decrease the levels of cationization; increase ion yields; increase precision of quantitation; increase sample-to-sample reproducibility and increase resolution.

The use of co-matrices is more common in the analysis of oligonucleotides, where ammonium salts and organic bases are very common additives. The addition of ammonium salts to the matrix-analyte solution has been shown to substantially enhance the signal for phosphopeptides. This has been used to allow the identification of phosphopeptides from unfractionated proteolytic digests. The approach works well with CHCA and DHB and with ammonium salts such as diammonium citrate and ammonium acetate some examples of additives used in peptide and protein measurements are bumetanide, glutathione, 4-nitro aniline, vanillin, nitrocellulose and L(−) fucose.

III. Solvent Selection

Solvent choice remains a trial-and-error process that is governed by the need to maintain analyte solubility and promote the partitioning of the analyte into the matrix crystals during drying of the analyte/matrix solution. It is preferable to first find the appropriate solvent for the sample and then choose a solvent for the matrix that is miscible with the analyte solvent. A 0.1% TFA solvent is preferred for peptides and proteins. The matrices for these analytes may be dissolved in CAN/0.1% TFA.

IV. Crystallization Methods

With minor modifications, the original dried droplet sample preparation procedure introduced by Hillenkamp and Karas (1988) has remained intact for over a decade.

It is important to find matrix molecules that will dry out of solution with analyte molecules in the resulting matrix crystals and that will enable the MALDI process. Poor sample preparation may result in low resolution, poor reproducibility and degraded sensitivity. MALDI optimization is primarily an empirical process that involves a significant amount of trial-and-error. Every choice during sample preparation can potentially affect the outcome of the MALDI measurement. It is not unusual to test a few different approaches before choosing the optimum protocol for sample preparation. The following are a variety of methods used for crystallization.

An aqueous solution of the matrix compound is mixed with analyte solution. A 1 mL droplet of this solution is then dried resulting in a solid deposit of analyte-doped matrix crystal that is introduced into the mass spectrometer for analysis. A general procedure is provided below.

1. Prepare a fresh saturated solution of matrix material in the solvent system of choice: A small amount, 10-20 mg, of matrix powder is thoroughly mixed with 1 mL of solvent in a 1.5 mL Eppendorf tube, and then centrifuged to pellet the undissolved matrix.

2. Place 5-10 mL of the saturated matrix solution in a small Eppendorf tube.

3. Add a smaller volume (1 to 2 mL) of protein solutions (1-100 mM) to the matrix.

4. Mix the solution thoroughly for a few seconds.

5. Place a 0.5-2 mL droplet of the resulting mixture on the mass spectrometer sample plate.

6. Dry the droplet at room temperature.

7. When the liquid has completely evaporated, the sample may be loaded into the mass spectrometer. Typical analyte amounts on MALDI crystalline deposits are in the 0.1-100 picomole range.

The vacuum-drying crystallization method is a variation of the dried-droplet method in which the final analyte/matrix drop applied to the sample stage is rapidly dried in a vacuum chamber. Vacuum drying is one of the simplest options available to reduce the size of the analyte-matrix crystals and increase crystal homogeneity by reducing the segregation effect. The general procedure is provided below.

1. Prepare the analyte/matrix sample solution following steps 1 through 4 of the dried-droplet method.

2. Apply a 0.5 to 2 mL drop of the solution to the sample stage.

3. Immediately introduce the sample stage into a vacuum-sealed container and pump the sample down to <10−2 Torr with a vacuum pump. Wait until the solvent is completely evaporated.

4. Introduce the sample into the mass spectrometer.

The crushed-crystal method was specifically developed to allow for the growth of analyte-doped crystals in the presence of high concentrations of involatile solvents such as glycerol, 6M urea and DMSO without purification. The general procedure is provided below.

1. A fresh saturated solution of matrix material in the solvent system of choice is prepared in the same fashion as in step 1 of the dried-droplet method. The supernatant liquid is transferred to a separate container before use to eliminate the potential presence of undissolved matrix crystals.

2. An aliquot (5 to 10 mL) of the saturated matrix solution is mixed with the protein containing solution (1 to 2 mL) to produce a final protein concentration of 0.1 to 10 mM. This analyte/matrix solution is equivalent to the one that would be made in the simpler dried-droplet method.

3. A 1 mL drop of the matrix-only solution is placed on the sample stage and dried in the air. The deposit formed looks identical to what is typically obtained from the dried-droplet deposit.

4. A clean glass slide is placed on the deposit and pressed down onto the surface with an elastic rod such as a pencil eraser. The glass surface is turned laterally several times to smear the deposit into the surface.

5. The crushed matrix is then brushed with a tissue to remove any excess particles.

6. A 1 mL droplet of the analyte/matrix solution is then applied to the spot bearing the smeared matrix material.

7. Within a few seconds an opaque film forms over the substrate surface covering the metal.

8. After about a minute the sample is immersed in room temperature water to remove involatile solvents and other contaminants.

9. The film is blotted with a tissue to remove excess water and allowed to dry before loading into the mass spectrometer.

The fast-evaporation method was introduced by Vorm et al. (Anal. Chem. 66:3281, 1994) with the main goal of improving the resolution and mass accuracy of MALDI measurements. It is a simple sample preparation procedure in which matrix and sample handling are completely decoupled.

The overlayer method was developed on the basis of the crushed-crystal method and the fast-evaporation method. It involves the use of fast solvent evaporation to form the first layer of small crystals, followed by deposition of a mixture of matrix and analyte solution on top of the crystal layer (as in the sample matrix deposition step of the crushed-crystal method). The origin of this method, and its multiple names can be traced back to the efforts of several research groups (Li et al., Anal. Chem. 71:5451, 1999; Li et al., Anal. Chem. 71:1087, 1999).

The sandwich method is derived from the fast-evaporation method and the overlayer method. It was reported for the first time by Li et al. (J. Am. Chem. Soc. 118:11662, 1996), and used for the analysis of single mammalian cell lysates by mass spectrometry.

The preparation of near homogeneous samples of large bimolecular, base on the method of spin-coating sample substrates was reported for the first time by Perera et al. (Rapid Commun. Mass Spectrom. 9:180, 1995).

It is possible to grow large, protein doped matrix crystals under near equilibrium conditions, rather than in a rapidly drying droplet (Beavis and Xiang, Org. Mass Spectrom. 28:1424, 1993). Supersaturated matrix solutions containing protein will form crystals that can be used directly in an ion source.

Electrospray as a sample deposition for MALDI-MS was suggested by Owens et al. and Axelsson et al. (Rapid Commun. Mass Spectrom. 11:209, 1997 and J. Am. Soc. Mass Spectrom. 10:1209, 1999, respectively). In this technique, a small amount of matrix-analyte mixture is electrosprayed from a HV-biased (3-5 kV) stainless steel or glass capillary onto a grounded metal sample plate, mounted 0.5-3 cm away from the tip of the capillary.

Early efforts with matrix precoated targets described the use of a pneumatic sprayer to fast-evaporate a thin matrix-only layer on a MALDI target (Kochling and Biemann, Proc. of the 43^{rd} Annual ASMS Conf. on Mass Spectrom. and Allied Topics, 1995). The microcrystalline films were stable and long-lived and provided adequate MALDI spectra for peptides and small proteins.

This method is faster and more sensitive than previously described methods and offers the opportunity to directly interface the MALDI sample preparation to the output of LC and CE columns.

C. Protein Digestion

Enzymatic cleavage and chemical cleavage are two commonly used methods for digesting proteins. In the present invention it is preferred that the digestion process cleave only at a specific amino acid or amino acid sequence, and cuts at all occurrences of that amino acid or amino acid sequence. Preferably the digestion of the protein does not produce so many fragments that separation becomes difficult correspondingly if too few fragments are produced they may be too produces peptides too large for certain kinds of analysis.

The most common digestions are with trypsin and lysine specific proteinases, because these enzymes are reliable, specific and produce a suitable number of peptides. The next most common digestion is at aspartate or glutamate using endoproteinase Glu-C or endoproteinase Asp-N. Chymotrypsin is sometimes used, although it does not have a well-defined specificity. Proteinases of broad specificity may generate many peptides and the peptides may be very short. Of the chemical cleavages, cyanogens bromide is the most common. All the chemical digestions are less efficient than a good enzymatic digest. However they do produce only a few peptides which can ease any purification problem.

D. MALDO-TOF Analysis

Direct analysis of diluted human whole blood by mass protein MassARRAY technology was successful in identifying α and β globin chains in normal Hb, and in thalassemia disease samples in adults. However only the ratio of α- and β-globin chains can be obtained; absolute quantification is not possible in the absence of an internal standard of known quantity, HbF γ-chains are also readily detectable in newborn samples, however the relatively small mass difference (14 Da) between Gγ and Aγ makes resolution, and especially accurate quantitation of the isoforms difficult.

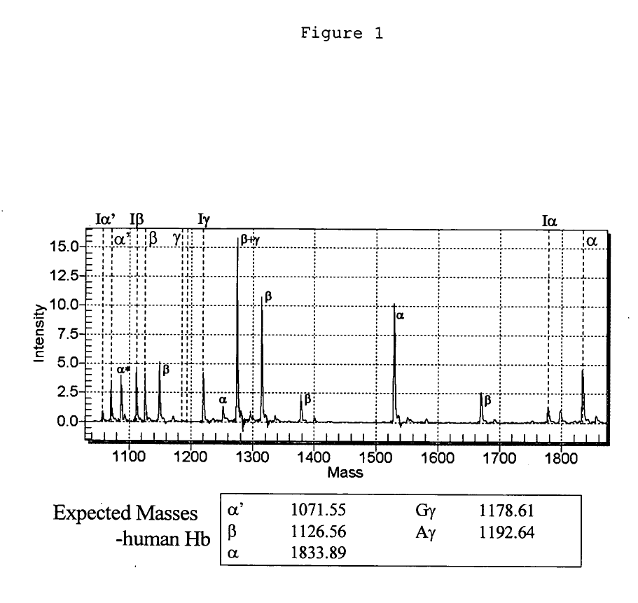

In order to reduce the size of the protein targets for easier and potentially more precise quantification, we have adopted the technique for peptide mass fingerprinting. The diluted blood samples are digested with the endoproteinase trypsin, which cleaves proteins specifically at the C-termini of lysine and arginine residues. Complete cleavage of the hemoglobin chains can be achieved with an overnight digestion. The resulting mixture of peptides in the mass range 500 to 3,500 Da is easily analyzed by MALDI-TOF. However reliable quantification of signature peptides from different chains is difficult since the relative intensity of the peptide peaks may vary between spots within a chip, ad between sample preparations. Examples are show in FIGS. 1A and B, which are spectra acquired from different spottings of the same sample. Except for the Gγ/Aγ ratio which is relatively constant, the ratio between each pair of peaks can be observed to vary. The differences in relative peak intensities may result from charge competition during MALDI ionization and local peptide and sale content in the matrix crystal. Another potential source of variation arises when comparing two peptides of very different amino acid sequence content, whose solubility in the analyte solution, degree of incorporation into the matrix crystal and ionization efficiency in MALDI could be very different. Compounded with the inhomogeneous crystallization of the sample/matrix spot, some variation in peak intensity ratios is anticipated. However if the compared peptides are very similar in both sequence content and length, such variation in peak intensity ratios can be minimized. In our experiments we have observed that the ratios of Gγ and Aγ, which differ by only one neutral amino acid (substitution of glycine to alanine), are more stable from spot-to-spot and sample-to-sample. This suggested that exogenous peptides similar in sequence and length to corresponding natural peptides may be expected to perform similarly in the MassARRAY, and if included in specific amounts could provide internal standards for precise quantification of the corresponding natural proteins (i.e. hemoglobin chains). A recent report demonstrated successful application of this approach to quantify the closely related human cardiac α- and β-myosin heavy chain (MyHC) proteins (Helmke et al., Anal. Chem. 2004, 76, 1683-1689). The authors first identified peptide pairs from the two MyHC isoforms with stable peak ratios. Then by introducing an internal standard peptide with one neutral amino acid substitution, simultaneous absolute quantification of both isoforms could be achieved.

1. A method to quantitate proteins associated with a disease or disease state comprising the steps of:
isolating at least two target proteins from a test sample;
combining said at least two target proteins with a known concentration of a concatenated oligopeptide standard and a proteinase forming peptide fragments and standard peptide fragments, said concatenated oligopeptide standard comprising at least one peptide having an amino acid sequence substantially similar to a unique amino acid sequence of a peptide fragment of said at least two target proteins, said at least one peptide for each of said at least two target proteins joined by proteinase cleavage sites to form said concatenated oligopeptide; and
quantitating said peptide fragments by comparison to said standard peptide fragments using mass spectroscopy.

2. A method to determine the efficiency of proteinase cleavage of proteins comprising the steps of:
combining a test sample containing target proteins with a known concentration of a concatenated oligopeptide standard and a proteinase forming peptide fragments and standard peptide fragments, said concatenated oligopeptide standard comprising at least one peptide having an amino acid sequence substantially similar to a unique amino acid sequence of a peptide fragment of said target proteins, said at least one peptide for each of said target proteins joined by proteinase cleavage sites to form said concatenated oligopeptide; and
determining the efficiency of said proteinase cleavage using mass spectroscopy by comparison of the amount of said standard peptide fragments have been cleaved by said proteinase to the amount of said standard peptide fragments that have not been cleaved by said proteinase.

3. A method to determine proteinase activity comprising the steps of:
combining a test sample containing target proteins with a known concentration of a concatenated oligopeptide standard and a proteinase for a set period of time forming peptide fragments and standard peptide fragments, said concatenated oligopeptide standard comprising at least one peptide having an amino acid sequence substantially similar to a unique amino acid sequence of a peptide fragment of said target proteins, said at least one peptide for each of said target proteins joined by proteinase cleavage sites to form said concatenated oligopeptide; and
determining the activity of said proteinase said standard peptide fragments using mass spectroscopy by comparison of the amount of said standard peptide fragments that have been cleaved by said proteinase to the amount of said standard peptide fragments that have not been cleaved by said proteinase during said set period of time.

4. A method according to claim 1 wherein said at least one peptide fragment from each of said at least two target proteins have different sequences.

5. A method according to claim 4 wherein said at least two peptide fragments of said concatenated oligopeptide standard are joined by at least one proteinase cleavage site in any order.

6. A method according to claim 1 wherein said concatenated oligopeptide standard comprises at least two standard peptide fragments each having a length not less than 9 amino acids.

7. A method according to claim 1 wherein said concatenated oligopeptide standard comprises at least two standard peptide fragments each having a length not more than 50 amino acids.

8. A method according to claim 1 wherein said proteinase cleavage site is a site that is cleaved by an proteinase selected from the group consisting of Lyc-C, trypsin, staphylococcus aureas V8 and Asp-N.

9. A method according to claim 1 wherein said at least two target proteins is a protein selected from the group consisting of antibodies, hemoglobin proteins, glycoprotein and enzymes.

10. A method according to claim 1 wherein said mass spectrometry is MALDI TOF mass spectrometry.

11. A concatenated oligopeptide comprising at least two peptides wherein each peptide has an amino acid sequence substantially similar to a unique amino acid sequence of a target protein said at least two peptides joined together by at least one proteinase cleavage sites.

12. A concatenated oligopeptide standard according to claim 11 wherein said at least two peptides each have a length not less than 9 amino acids.

13. A concatenated oligopeptide standard according to claim 11 wherein said at least two peptides each have a length not more than 50 amino acids.

14. A concatenated oligopeptide standard having the following sequence:
MFVSFPTTKVHVDPENFRMVTVVASALSSRTYFPHFDGSHGSAQVK (SEQ ID NO.1).

15. A concatenated oligopeptide standard according to claim 11 comprising at least two peptide fragments wherein at least one of said peptide fragments is labeled with an isotope.

16. A concatenated oligopeptide standard according to claim 15 wherein said isotope label is ¹⁵N, ¹³C or ¹⁸O.

17. A concatenated oligopeptide standard according to claim 15 wherein said at least two peptides each have a length not less than 9 amino acids.

18. A concatenated oligopeptide standard according to claim 15 wherein said at least two peptides each have a length not more than 50 amino acids.


SEQUENCE LISTING (18 sequences)

SEQ ID NO: 1 - protein, 46 aa, Homo sapiens
MFVSFPTTKVHVDPENFRMVTVVASALSSRTYFPHFDGSHGSAQVK

SEQ ID NO: 2 - protein, 37 aa, Homo sapiens
VHVDPENFRMVTVVASALSSRTYFPHFDGSHGSAQVK

SEQ ID NO: 3 - protein, 28 aa, Homo sapiens
MVTVVASALSSRTYFPHFDGSHGSAQVK

SEQ ID NO: 4 - protein, 30 aa, Homo sapiens
MFVSFPTTKVHVDPENFRMVTVVASALSSR

SEQ ID NO: 5 - protein, 18 aa, Homo sapiens
MFVSFPTTKVHVDPENFR

SEQ ID NO: 6 - protein, 12 aa, Homo sapiens
MVTVVASALSSR

SEQ ID NO: 7 - protein, 9 aa, Homo sapiens
VHVDPENFR

SEQ ID NO: 8 - protein, 16 aa, Homo sapiens
TYFPHFDLSHGSAQVK

SEQ ID NO: 9 - protein, 9 aa, Homo sapiens
MFVSFPTTK

SEQ ID NO: 10 - protein, 12 aa, Homo sapiens
MVTGVASALSSR

SEQ ID NO: 11 - protein, 12 aa, Homo sapiens
MVTAVASALSSR

SEQ ID NO: 12 - protein, 9 aa, Homo sapiens
LHVDPENFR

SEQ ID NO: 13 - protein, 16 aa, Homo sapiens
TYFPHFDGSHGSAQVK

SEQ ID NO: 14 - protein, 9 aa, Homo sapiens
MFLSFPTTK

SEQ ID NO: 15 - protein, 16 aa, Homo sapiens
ILNPVAIPEGQFIDSR

SEQ ID NO: 16 - protein, 16 aa, Homo sapiens
ILNPAAIPEGQFIDSR

SEQ ID NO: 17 - protein, 32 aa, Homo sapiens
ILNPVAVPEGQFIDSRILNPAAVPEGQFIDSR

SEQ ID NO: 18 - protein, 21 aa, Homo sapiens
VHVDPENFRMVTVVASALSSR